(19) United States
(12) Patent Application Publication
(10) Pub. No.: US 2026/0085346 A1
(43) Pub. Date: Mar. 26, 2026

(54) METHODS AND SYSTEMS OF DETERMINING OPTIMAL AMPLIFICATION CYCLE NUMBERS

(71) Applicant: LIFE TECHNOLOGIES CORPORATION, Carlsbad, CA (US)

(72) Inventors: I-Jane Chen, Pleasanton, CA (US); Harrison Leong, San Francisco, CA (US); Siddhita Gopinath, Fremont, CA (US)

(21) Appl. No.: 19/326,603

(22) Filed: Sep. 11, 2025

Related U.S. Application Data
(60) Provisional application No. 63/698,487, filed on Sep. 24, 2024.

(51) Int. Cl. C12Q 1/6848

(52) U.S. Cl. CPC C12Q 1/6848

(57) ABSTRACT

Provided herein are methods and systems of determining an optimal amplification cycle number during an amplification reaction of a template with unknown concentrations, thereby generating an amplification output within a narrow concentration range and desired quantities regardless of its initial template input. The optimal amplification cycle number can be determined based on a transition point corresponding to a change in signals of the amplification reaction. The transition point can be determined based on applying a derivative to the signals of the amplification, determining a baseline of the signals, or a combination thereof.

### CROSS-REFERENCE TO RELATED APPLICATIONS

This application claims priority under 35 U.S.C. 120 to U.S. Provisional Application No. 63/698,487, filed Sep. 24, 2024. The disclosure of the above-identified application is incorporated herein by reference in its entirety for all purposes.

### BACKGROUND

A polymerase chain reaction (PCR) is an in vitro method for enzymatically synthesizing or amplifying defined nucleic acid sequences. The reaction typically uses two oligonucleotide primers that hybridize to opposite strands and flank a template or target deoxyribonucleic acid (DNA) sequence that is to be amplified. Elongation of the primers is catalyzed by a heat-stable DNA polymerase. A repetitive series of cycles involving template denaturation, primer annealing, and extension of the annealed primers by the polymerase results in an exponential accumulation of a specific DNA fragment. Fluorescent probes or markers are typically used in the process to facilitate detection and quantification of the amplification process.

Performing PCR can involve setting a PCR cycle number based on a DNA template quantity in each sample being amplified. Typically, for each sample, there is a range of cycle numbers within which suitable amplification can occur. A cycle number that is too high can result in over-amplification which increases nonspecific hybridization among different PCR products and/or by-products. Conversely, a cycle number that is too low can result in under-amplification such that users need to perform one or more additional iterations of PCR. Both over-amplification and under-amplification can compromise a quantity of amplified DNA, causing the amplified DNA to be unanalyzable.

A suboptimal cycle number can refer to a cycle number outside of the range of cycle numbers corresponding to suitable amplification. Using a suboptimal cycle number to perform PCR may not only compromise the quantity of the final DNA (e.g., resulting in a poor detection limit), but also produce PCR end results with poor quality such that the PCR end results are not analyzable. In addition, determining a DNA template number prior to setting up a PCR run is time consuming, expensive, and adds extra workload on scientists or other users of PCR. As such, a new methodology is needed that can remove such burdens from day-to-day lab work, while also creating the possibility of building a truly sample-to-answer capability for technologies that utilize DNA.

### BRIEF SUMMARY

The present disclosure relates generally to systems and methods for sensing DNA amplification at real time to predict an optimal cycle number for performing a polymerase chain reaction (PCR).

In one aspect, the present disclosure provides a method of determining an optimal amplification cycle number m for an amplification reaction of a template nucleic acid of unknown copy number in a sample. In some embodiments, the method comprises detecting signals representing amplification products at each of the n cycles of the amplification reaction; monitoring, based on the detected signals, the amplification reaction as a function of cycle number; determining a transition point of the amplification reaction, wherein the transition point corresponds to a change in the detected signals; and predicting the optimal cycle number m based on the transition point, wherein m is larger than n.

In some embodiments, the method further comprises determining an initial baseline based on a subset of the detected signals corresponding to a predefined number of the n cycles; in response to receiving an updated set of the detected signals, comparing the updated set of the detected signals to the initial baseline; if the updated set of the detected signals is consistent with the initial baseline, updating the initial baseline to include the updated set of the detected signals; and if the updated set of the detected signals exceeds the initial baseline, determining that the transition point corresponds to a particular cycle number of the updated set of the detected signals. In some embodiments, the initial baseline is determined based on one or more patterns in a zeroth derivative, a first derivative, and/or a second derivative of the detected signals. In some embodiments, the initial baseline is determined by a machine learning approach or an artificial intelligence approach.

In some embodiments, the transition point is determined based on a zeroth derivative, a first derivative, and/or a second derivative of the detected signals. In some embodiments, the transition point corresponds to a peak of the second derivative of the detected signals. In some embodiments, the transition point corresponds to the zeroth derivative, the first derivative, and/or the second derivative of the function exceeding a set of one or more thresholds for each derivative type. In some embodiments, the transition point corresponds to the second derivative of the function exceeding a threshold, and wherein a third derivative changes sign. In some embodiments, the transition point is determined when an amplification efficiency rate determined from the detected signals is consistently positive and within an expected range.

In some embodiments, the optimal amplification cycle number m is determined by adding an adjustment value to a cycle number corresponding to the transition point of the amplification. In some embodiments, the adjustment value is predefined based on the determination of a transition point. In some embodiments, m is equal to or more than n+1. In some embodiments, n is an integer at least 1.

In some embodiments, the template nucleic acid has a concentration in a range of 0.001-500 ng per 25 μL, optionally in a range of 0.1-100 ng per 25 μL. In some embodiments, the amplification reaction is a Taqman PCR.

In some embodiments, the predicting step comprises providing the detected signals as input to an artificial intelligence (AI) model, wherein the AI model optionally comprises a machine-learning model; and executing, based on the detected signals, the AI model to generate an output indicating the optimal cycle number.

In some embodiments, prior to the detecting step, the method further comprises training a computer program using one or more training samples with known optimal cycle numbers. In some embodiments, the optimal cycle number m is predicted by the computer program using one or more cycles of the detected signals.

In another aspect, the present disclosure provides a method of amplifying one or more target genes in a sample. In some embodiments, the method comprises performing an amplification reaction using the sample; determining an optimal amplification cycle number m using the method described herein, and stop the amplification reaction at the optimal amplification cycle number m. In some embodiments, the amplification reaction comprises amplifying a housekeeping gene simultaneously with the one or more target genes in the same sample.

In another aspect, the present disclosure provides a computer product comprising a non-transitory computer readable medium storing a plurality of instructions to perform operations that, when executed, control a computer system determine an optimal amplification cycle number m for an amplification reaction of a template nucleic acid of unknown copy number in a sample. In some embodiments, the operations comprise detecting signals representing amplification products at each of the n cycles of the amplification reaction; monitoring, based on the detected signals, the amplification as a function of cycle number; determining a transition point of the amplification, wherein the transition point corresponds to a change in the detected signals; and predicting the optimal cycle number m based on the transition point, wherein m is larger than n.

In some embodiments, the operations further comprise determining an initial baseline based on a subset of the detected signals corresponding to a predefined number of the n cycles; in response to receiving an updated set of the detected signals, comparing the updated set of the detected signals to the initial baseline; if the updated set of the detected signals is consistent with the initial baseline, updating the initial baseline to include the updated set of the detected signals; and if the updated set of the detected signals exceeds the initial baseline, determining that the transition point corresponds to a particular cycle number of the updated set of the detected signals. In some embodiments, the initial baseline is determined based on one or more patterns in a zeroth derivative, a first derivative, and/or a second derivative of the detected signals. In some embodiments, the initial baseline is determined by a machine learning approach or an artificial intelligence approach.

In some embodiments, the transition point is determined based on a zeroth derivative, a first derivative, and/or a second derivative of the function. In some embodiments, m is determined by adding an adjustment value to a cycle number corresponding to the transition point of the curve. In some embodiments, the adjustment value is predefined based on the determination of the transition point.

In another aspect, the present disclosure provides a Polymerase Chain Reaction (PCR) system. In some embodiments, the PCR system comprises a PCR data acquiring device configured to detect signals representing amplification products at each of the n cycles of the amplification reaction; and a computer system configured to process the signals to determine an optimal amplification cycle number m for an amplification reaction of a template nucleic acid of unknown copy number in a sample. In some embodiments, the computer system comprises instructions to perform operations. The operations comprise receiving the detected signals; monitoring, based on the detected signals, the amplification reaction as a function of cycle number; determining a transition point of the amplification reaction, wherein the transition point corresponds to a change in the detected signals; and predicting the optimal cycle number m based on the transition point, wherein m is larger than n.

In some embodiments, the transition point is determined by determining an initial baseline based on a subset of the detected signals corresponding to a predefined number of the n cycles; in response to receiving an updated set of the detected signals, comparing the updated set of the detected signals to the initial baseline; if the updated set of the detected signals is consistent with the initial baseline, updating the initial baseline to include the updated set of the detected signals; and if the updated set of the detected signals exceeds the initial baseline, determining that the transition point corresponds to a particular cycle number of the updated set of the detected signals. In some embodiments, the initial baseline is determined based on one or more patterns in a zeroth derivative, a first derivative, and/or a second derivative of the detected signals. In some embodiments, the initial baseline is determined by a machine learning approach or an artificial intelligence approach. In some embodiments, the transition point is determined based on a zeroth derivative, a first derivative, and/or a second derivative of the function.

A better understanding of the nature and advantages of the present invention may be gained with reference to the following detailed description and the accompanying drawings.

### BRIEF DESCRIPTION OF THE DRAWINGS

The foregoing aspects and many of the attendant advantages of the present disclosure will become more readily appreciated as the same become better understood by reference to the following detailed description, when taken in conjunction with the accompanying drawings.

FIG. 1A illustrates an example of a typical real time PCR with fixed thermocycle numbers, where a resulting final amplicon concentration falls outside of the suitable dynamic range of sensors.

FIG. 1B illustrates an example of a sensing multiplication at real time (SMaRT) PCR, resulting final amplicon concentration falls within a suitable dynamic range of sensors.

FIG. 2 is a flowchart illustrating a method of determining an optimal amplification cycle number for an amplification reaction of a template nucleic acid of unknown copy number in a sample.

FIG. 3A illustrates PCR growth curves of the DNA templates with 10-fold serial dilutions in a TaqMan™ assay.

FIG. 3B illustrates PCR growth curves of the DNA templates with 2-fold serial dilutions in a TaqMan™ assay.

FIG. 4A illustrates an example of sensing algorithm training for prediction of optimal cycle number of a real time amplification, resulting Algorithm 1, CtProxy, that calls an optimal cycle number 1.5 (+/−0.5) cycles ahead of time.

FIG. 4B illustrates an example of sensing algorithm training for prediction of optimal cycle number of a real time amplification, resulting Algorithm 2, EffLoc, that calls an optimal cycle number 5 (+/−1) cycles ahead of time.

FIG. 5 illustrates equivalent cycle number prediction between replicates of the same samples of within +/−1 cycle.

FIG. 6 illustrates equivalent average sample peak heights between replicates of the same samples of within +/−1 cycle.

FIG. 7 is a flow chart of an example method of determining an optimal amplification cycle number according to some embodiments of the present disclosure.

FIG. 8 is a block diagram of an example computer system 800 usable with systems and methods according to some embodiments of the present disclosure.

In the drawings, like reference numerals refer to like parts throughout the various views unless otherwise specified. Not all instances of an element are necessarily labeled to reduce clutter in the drawings where appropriate. The drawings are not necessarily to scale, emphasis instead being placed upon illustrating the principles being described.

### DETAILED DESCRIPTION

Embodiments of the present invention provide methods and systems for determining an optimal amplification cycle number for an amplification reaction, such as, from a template nucleic acid of unknown copy number in a sample. The methods and systems can apply to a wide range of samples (such as blood spot samples, buccal swab samples, etc.) wherein the samples contain a wide range of DNA concentrations. The methods and systems can sense, calculate and determine the best amplification cycle number for each sample, assuring the final PCR products fall within a narrow, desired quantity range regardless of its initial sample input.

While exemplary embodiments have been illustrated and described, it will be appreciated that various changes can be made therein without departing from the spirit and scope of the disclosure.

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Numeric ranges recited within the specification are inclusive of the numbers within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

Also, the words “comprise,” “comprising,” “contains,” “containing,” “include,” “including,” and “includes,” when used in this specification and in the following claims, are intended to specify the presence of stated features, integers, components, or steps, but they do not preclude the presence or addition of one or more other features, integers, components, steps, acts, or groups.

### I. GENERAL OVERVIEW

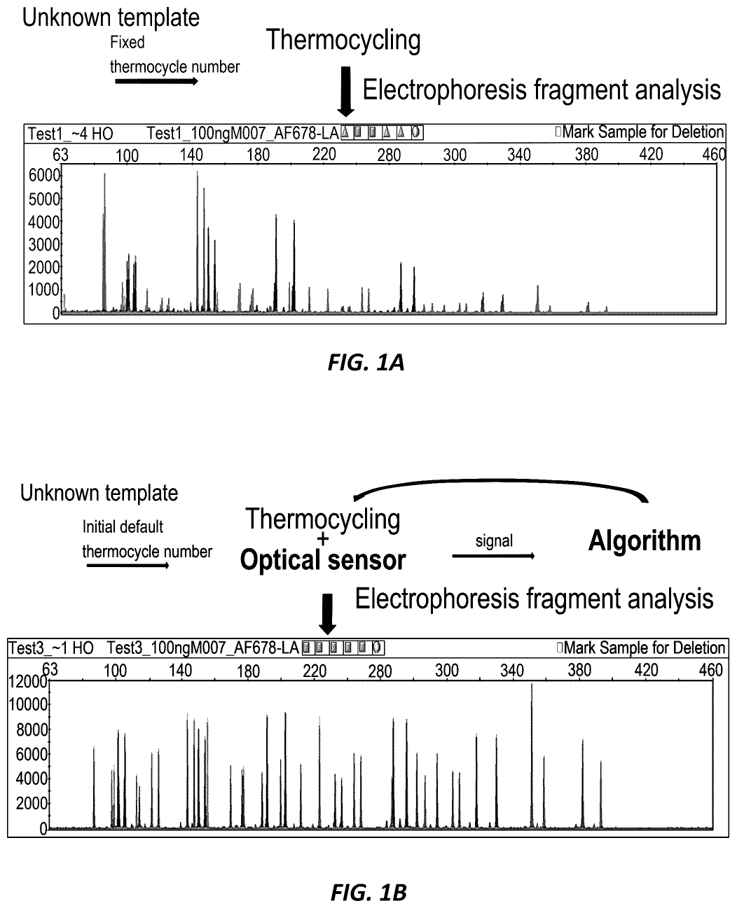

The present disclosure provides a method that assures the quality and quantity of PCR amplicons without performing quality and quantity tests prior to, in the middle of, or at the end of PCR reactions. Conventional PCR amplification mechanisms can involve a user inputting an initial guess of an optimal cycle number to perform PCR. The initial guess may result in an unsuitable amount of PCR amplicons that is outside of a desired range (e.g., too high or too low in quantity), as illustrated in FIG. 1A. Additionally, the user may perform additional steps, such as quantifying the amplicons after an iteration of PCR or performing another iteration of PCR, based on PCR results after implementing the initial guess. The method of determining an optimal amplification cycle number described herein implements an automated PCR mechanism that produces PCR amplicons within the desired range based on the optimal amplification number despite an unknown amount of sample input, as illustrated in FIG. 1B. This method utilizes a real time DNA quantity sensing module and a computational algorithm to predict optimal amplification cycle numbers of the PCR reactions.

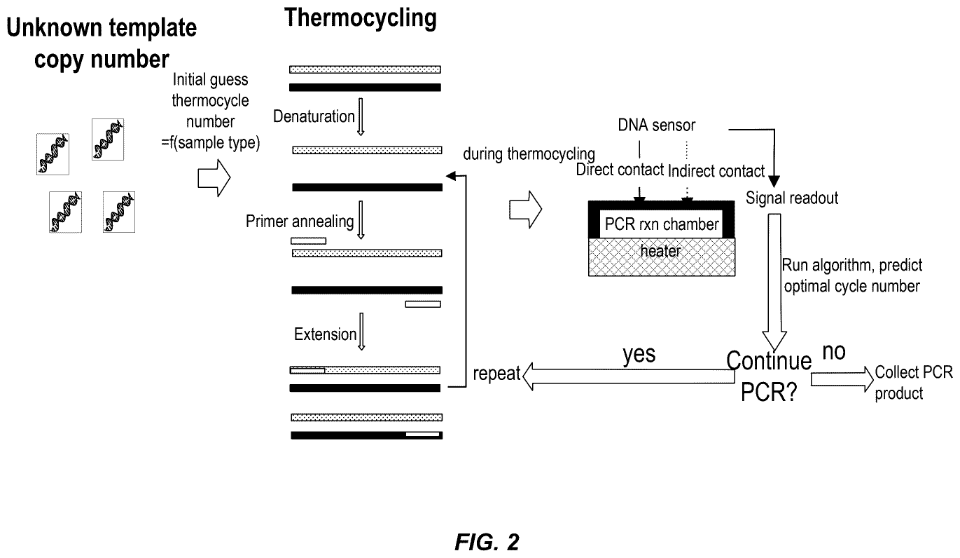

As illustrated in FIG. 2, the method includes amplifying a nucleic acid template with an unknown copy number from a sample. One or more sensors are placed at a PCR reaction chamber, directly or indirectly, in contact with the reagents. As PCR reactions take place, the real time DNA quantity signals are caught by the sensors and sent to a computing device configured to apply an algorithm to determine an optimal amplification cycle number. After a few cycles (e.g., about 1˜20 cycles, optionally about 5˜10 cycles) of collecting signals, the algorithm performs a mathematical calculation and provides an optimal cycle number for each run. The PCR reaction is then terminated at the optimal cycle number provided by the algorithm, resulting in a desired range of DNA quantity.

In one aspect, the present disclosure provides a method of determining an optimal amplification cycle number m for an amplification reaction of a template nucleic acid of unknown copy number in a sample. In some embodiments, the method comprises detecting signals representing amplification products at each of the n cycles of the amplification reaction; monitoring, based on the detected signals, the amplification as a function of cycle number; determining a transition point of the amplification, wherein the transition point corresponds to a change in the detected signals; and predicting the optimal cycle number m based on the transition point, wherein m is larger than n.

In some embodiments, the method further comprises determining an initial baseline based on a subset of the detected signals corresponding to a predefined number of the n cycles; in response to receiving an updated set of the detected signals, comparing the updated set of the detected signals to the initial baseline; if the updated set of the detected signals is consistent with the initial baseline, updating the initial baseline to include the updated set of the detected signals; and if the updated set of the detected signals exceeds the initial baseline, determining that the transition point corresponds to a particular cycle number of the updated set of the detected signals.

In some embodiments, the optimal amplification cycle number m is determined by adding an adjustment value to a cycle number corresponding to the transition point of the amplification. In some embodiments, the adjustment value is predefined based on the determination of the transition point. In some embodiments, m is equal to or more than n+1. In some embodiments, n is an integer at least 1.

In some embodiments, the predicting step comprises providing the detected signals as input to an artificial intelligence (AI) model, wherein the AI model optionally comprises a machine-learning model; and executing, based on the detected signals, the AI model to generate an output indicating the optimal cycle number.

In some embodiments, prior to the detecting step, the method further comprises training a computer program using one or more training samples with known optimal cycle numbers. In some embodiments, the optimal cycle number m is predicted by the computer program using one or more cycles of the detected signals.

In another aspect, the present disclosure provides a method of amplifying one or more target genes in a sample. In some embodiments, the method comprises performing an amplification reaction using the sample; determining an optimal amplification cycle number m using the method described herein, and stop the amplification reaction at the optimal amplification cycle number m. In some embodiments, the amplification reaction comprises amplifying a housekeeping gene simultaneously with the one or more target genes in the same sample.

In another aspect, the present disclosure provides a computer product comprising a non-transitory computer readable medium storing a plurality of instructions to perform operations that, when executed, control a computer system to determine an optimal amplification cycle number m for an amplification reaction of a template nucleic acid of unknown copy number in a sample. In some embodiments, the operations comprise detecting signals representing amplification products at each of the n cycles of the amplification reaction; monitoring, based on the detected signals, the amplification as a function of cycle number; determining a transition point of the amplification, wherein the transition point corresponds to a change in the detected signals; and predicting the optimal cycle number m based on the transition point, wherein m is larger than n.

The present disclosure also provides a system (e.g., a PCR system) that senses, calculates and determines an optimal cycle number for each sample. The system disclosed herein assures the final PCR product falls within a narrow, desired quantity range regardless of its initial sample input. In some embodiments, the PCR system comprises a PCR data acquiring device configured to detect signals representing amplification products at each of the n cycles of the amplification reaction; and a computer system described herein configured to process the signals to determine an optimal amplification cycle number m for an amplification reaction of a template nucleic acid of unknown copy number in a sample.

The methods and systems disclosed herein can apply to broad sample types and wide range of DNA templates. For example, the methods and systems can apply to human identification (HID) system to process broader sample types that inherently have wider range of DNA templates to be amplified and analyzed. The methods and systems can also apply to the life science field where a pre-amplification of samples is required, including but not limited to, digital PCR (dPCR), quantitative PCR (qPCR), capillary electrophoresis (CE) and next gen sequencing (NGS).

As disclosed herein, an optimal amplification cycle number is associated with sample input. A higher concentration sample input results in a smaller cycle number (or a shorter amplification reaction). A lower concentration sample input results in a bigger cycle number (or a longer amplification reaction). In some embodiments, one amplification reaction can comprise a mixture of templates to amplify a group of genes of interest.

The methods disclosed herein can be used to amplify DNAs from a variety of sources or samples. In some embodiments, the methods and systems can be used for human identification (HID) from a variety of biological samples such as blood samples, plasma samples, urine samples, tissue samples, saliva samples, buccal swab samples, etc.

The template can be one or more nucleic acid DNA fragments with unknown concentrations in a sample. As such, the methods and systems can be used to amplify DNAs from a sample without pre quantifying the sample before the PCR or interrupting the PCR to verify the quality of amplicons. By implementing the methods and systems disclosed herein, a streamlined workflow from sample amplification to sample analysis can be done in a fully automated and integrated process.

The methods and systems can be also used to amplify DNAs from a template with a wide range of DNA concentrations. In some embodiments, the template nucleic acid has a concentration in a range of 0.001-1000 ng, 0.001-500 ng, 0.1-500 ng, 0.1-100 ng in a 10-100 μL amplification reaction. In some embodiments, the template nucleic acid has a concentration in a range of about 0.001-500 ng in a 25 μL amplification reaction. In some embodiments, the template nucleic acid has a concentration in a range of 0.1-100 ng per 25 μL.

The methods and systems disclosed herein can monitor amplicon quality real time and predict optimal cycle numbers during the amplification reaction. Unlike a traditional amplification reaction (such as a real time PCR), no calibration curve prior the amplification reaction is needed using the methods and systems disclosed herein.

In the beginning of the amplification reaction, signals representing amplification products at each amplification cycle are detected by one or more sensors. As shown in FIG. 2, the methods and systems disclosed herein can be used to amplify a nucleic acid template with unknow copy number from a sample. During thermocycling, DNA sensors, directly or indirectly contacted with a PCR reaction chamber, can detect amplification signals in real time. The signals are then analyzed by a preset algorithm which provides an optimal cycle number of the sample during the amplification with a few cycles lead time (such as 1-20 cycles) and send to a command center for a go/no go of PCR cycling. In some embodiments, the preset algorithm is trained using known optimal cycle numbers on a range of sample types and sample concentrations to predict optimal cycle numbers of samples with unknown template concentrations. Non-limiting examples of the sample types include buccal swabs, dry blood, liquid blood, plasma, urine, saliva, and tissue samples.

In some embodiments, the amplification reaction is a polymerase chain reaction (PCR). In some embodiments, the amplification reaction is a digital PCR (dPCR), quantitative PCR (qPCR), capillary electrophoresis (CE) or next gen sequencing (NGS). In some embodiments, amplification reaction is a real time qPCR. In some embodiments, the amplification reaction is a Taqman PCR.

As disclosed herein, the amplification signals (either double or single stranded DNA) can be detected through a sensor. The term “sensor” refers to a device that detects and responds to a specific input, such as light, temperature, pressure, or motion and converts it into a measurable output. In some embodiments, a sensor can be also a transducer of any mechanism that converts one form of energy into another, such as converting mechanical energy into electrical signals.

The sensor disclosed herein can be an optical, electrical, electrochemical, chemical, mechanical sensor. In some embodiments, an optical sensor has one or more features including, but not limited to, direct UV absorption, directly labeling DNA using dyes (DAPI, SYBR, YOYO), surface plasmon resonance (SPR), Förster resonance energy transfer (FRET) and Taqman. In some embodiments, a mechanical sensor comprises quartz crystal microbalance. In some embodiments, an electrochemical sensor has one or more features including, but not limited to, cyclic voltammetry, conductivity, amperometry, and/or impedance.

As disclosed herein, the sensor can directly or indirectly contact a PCR reaction chamber (see. FIG. 2). In some instances, the sensor or transducer is embedded directly on a sample cartridge. In other instances, the sensor or transducer is embedded on an instrument near a PCR reaction chamber.

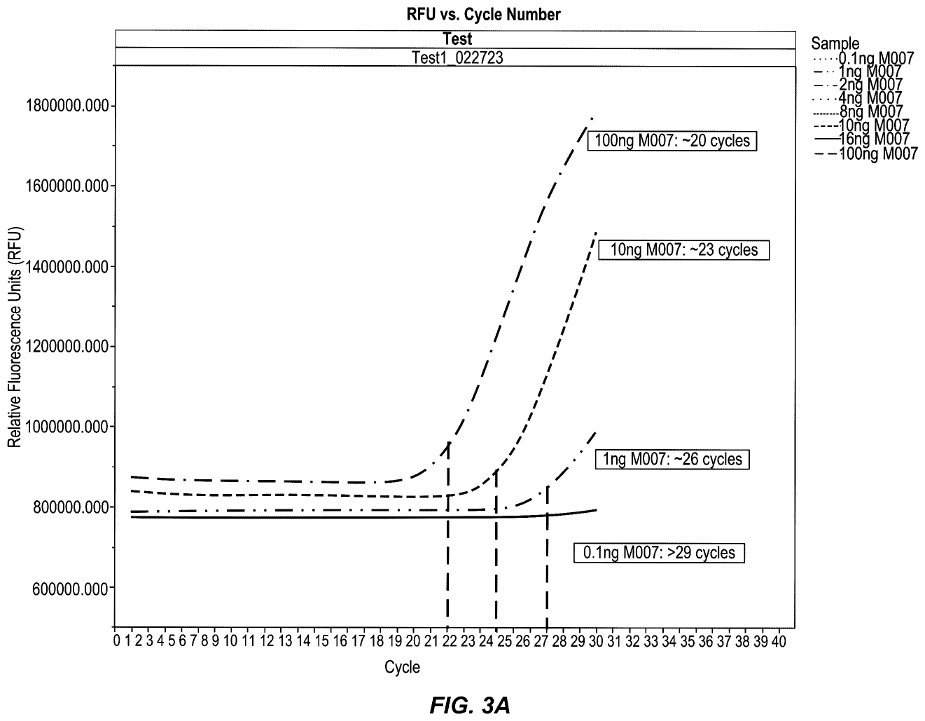

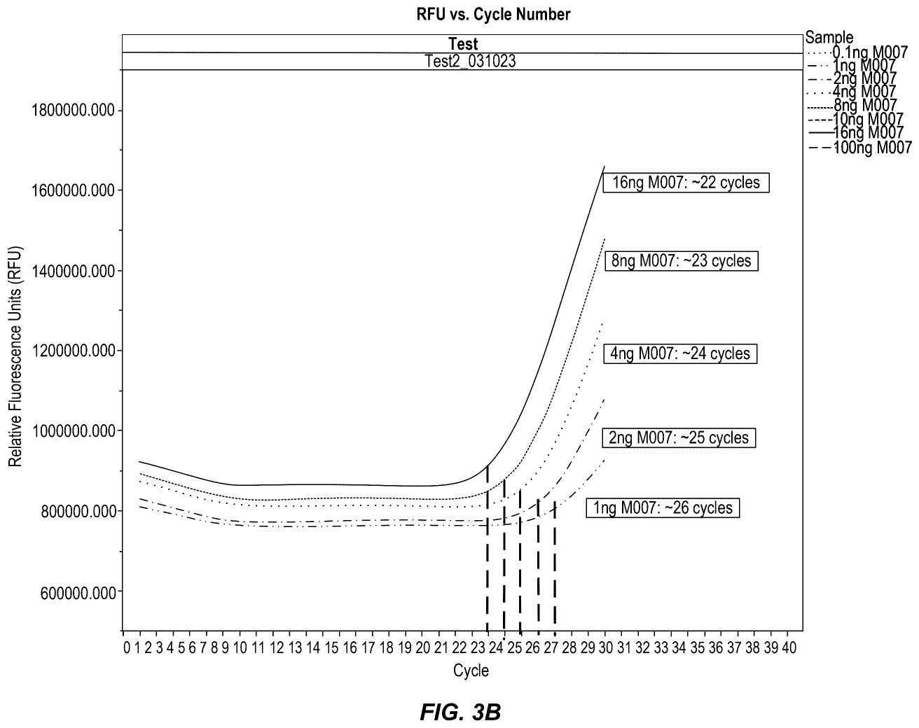

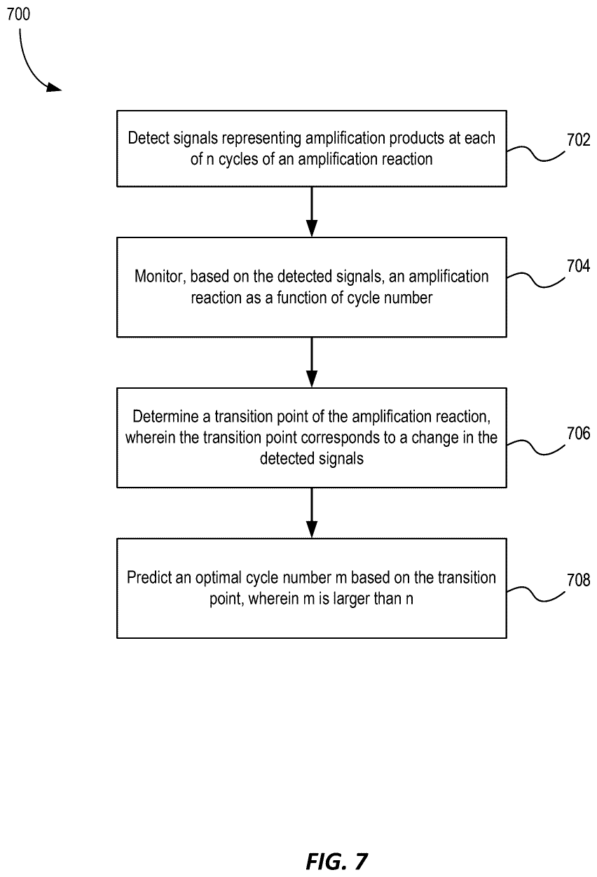

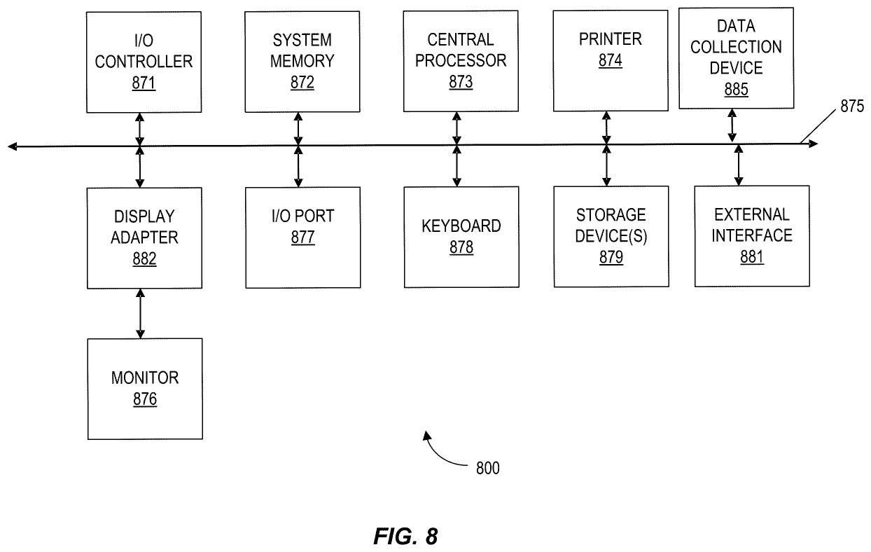

As described herein, the amplification signals collected by the sensor can indicate an amount of amplification product corresponding to a particular cycle number. FIG. 3A and FIG. 3B illustrate example PCR growth curves of DNA templates with 10-fold serial dilutions or 2-fold serial dilutions, respectively. The example PCR growth curves can be generated (e.g., plotted or otherwise visualized) based on amplification signals collected by the sensor. As shown, the example PCR growth curves correspond to functions of amplification signal (y-axis) versus cycle number (x-axis). More specifically, the amplification signals shown in FIG. 3A and FIG. 3B correspond to fluorescence signal strength (in relative fluorescence units (RFUs)) of a fluorescent indicator probe detected using an optical sensor. Additional details related to FIG. 3A and FIG. 3B are provided below with respect to FIG. 7 and in Example 2. In some embodiments, the amplification signals may undergo pre-processing (e.g., noise reduction) prior to analysis or visualization.

As disclosed herein, the optimal cycle number of an amplification reaction is determined based on a transition point of the amplification reaction. The transition point usually occurs one or a few cycles ahead of the optimal cycle number. As described herein, a derivative of a dataset including signals of the amplification reaction can indicate a rate of change with respect to the detected signals as well as the amplification reaction. An initial portion of the amplification reaction can correspond to a region (e.g., a baseline region) where a magnitude of the detected signals is relatively consistent and relatively low. As shown in FIG. 3A and FIG. 3B, the baseline region of the amplification reaction can be relatively linear or flat, indicating that little to no changes are detected in the signals of the amplification reaction. The initial portion of the amplification reaction can correspond to data from initial amplification cycles before amplification is evident. For example, within the initial portion of the amplification reaction, signals from a fluorescent probe may not have accumulated sufficiently to be visible. At or past the transition point, an amplification rate of the amplification reaction can increase above a predefined threshold. In some examples, the amplification rate may increase exponentially at or past the transition point. Accordingly, the amplification reaction may enter an exponential phase with exponential growth at or past the transition point.

In certain aspects, an algorithm (e.g., a computer program) can be provided to determine the optimal cycle number at which to stop the amplification reaction. As a non-limiting example, the algorithm may be implemented as a statistical model, such as a regression model. In other examples, the algorithm may be implemented as part of an artificial intelligence (AI) model (e.g., a machine-learning model) or a predictive model. In some examples, the algorithm can be trained prior to being implemented, such as being executed, downloaded, or otherwise accessed via a computer system (e.g., computer system 800). The algorithm can be trained using a set of training samples with known optimal cycle numbers. The set of training samples can include cycle-by-cycle data of one or more PCR runs, derivatives of the cycle-by-cycle data, known optimal cycle numbers, or a combination thereof. In some cases, the set of training samples can be prepared at least in part based on historical PCR runs. In some examples, training the algorithm can involve determining a signal change corresponding to a desired amount of amplicons produced using the amplification reaction. In some implementations, the algorithm can be trained in a supervised manner or an unsupervised manner. In supervised training, each input of the set of training samples can be correlated to a desired output, enabling the algorithm to determine a mapping between the inputs of the set of training samples and desired outputs. For example, cycle-by-cycle data of the set of training samples can be correlated to a corresponding known optimal cycle number. In unsupervised training, the set of training samples may include inputs but not desired outputs, such that the algorithm determines patterns or structure in the inputs on its own.

In some examples, the algorithm may be trained on a corpus of amplification reaction (e.g., PCR) data. The corpus can be a collection of sample data organized into one or more datasets. In particular, the corpus can include amplification reaction data related to samples where there is detectable amplification. Additionally, the corpus can include amplification reaction data related to samples where there is no detectable amplification. Accordingly, it would be possible to use deep learning, reinforcement learning, competitive learning, or other suitable machine learning techniques that can result in algorithms adept at distinguishing amplified signals from non-amplified signals, which in turn, can be used to identify the transition point. For example, if the algorithm is trained using the corpus that includes respective datasets corresponding to detectable amplification and no detectable amplification, the algorithm can be finetuned or otherwise adjusted based on the datasets of the corpus such that the algorithm can determine the transition point using previously unseen data. In particular, determining the signal change of the amplification reaction can involve distinguishing the amplified signals from the non-amplified signals.

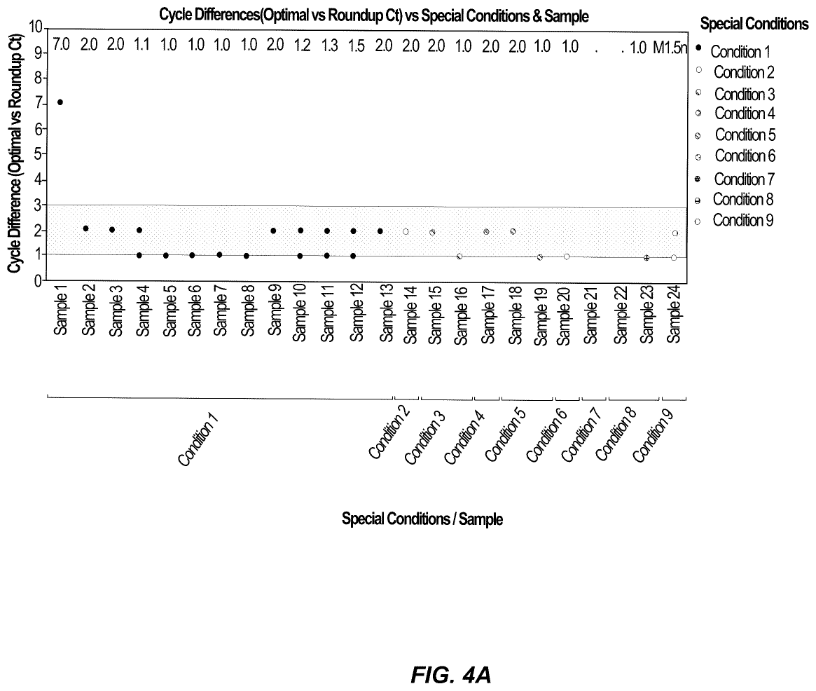

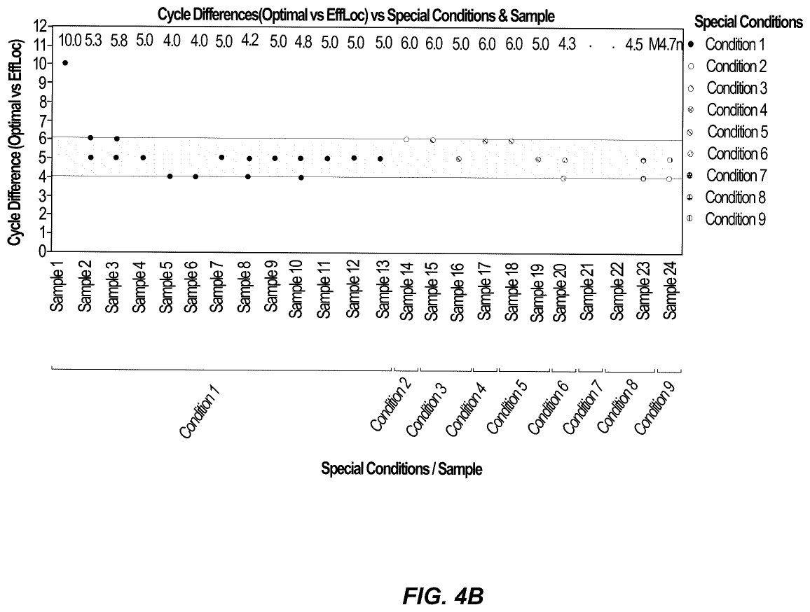

FIG. 4A and FIG. 4B illustrate examples of sensing algorithm training to predict an optimal cycle number of a real-time amplification reaction. In particular, FIG. 4A and FIG. 4B provide plots indicating differences (y-axis) in a breakout cycle number and a known optimal cycle number for each sample (x-axis) shown. Results shown in FIG. 4A and FIG. 4B were generated using different versions of the algorithm. More specifically, for FIG. 4A, the algorithm is shown to generate a consistent difference of about 1.5 cycles (plus or minus 0.5 cycles) between the breakout cycle number and the optimal cycle number. For FIG. 4B, its algorithm is shown to generate a consistent difference of about 5 cycles (plus or minus 0.5 cycles) between the breakout cycle number and the optimal cycle number. A total of 24 samples were tested using PCR to determine the differences in the breakout cycle number and the known optimal cycle number. Examples of sample types tested include blood swabs or buccal swabs. Nine different conditions were used to prepare the samples to determine whether different conditions affect a consistency of the differences between the breakout cycle number and the known optimal cycle number. For example, different reaction volumes (e.g., 7 μL versus 15 μL) were tested using the same sample type. As another example, different concentrations or amounts of inhibitors (e.g., humic acid, hematin, soil, etc.) were added to prepare and compare samples of the same sample type. Additional details related to FIG. 4A and FIG. 4B are provided below in Example 3.

The breakout cycle number corresponds to a cycle number of the transition point, such as where an exponential phase of amplification begins to be observable by the system. The difference between the breakout cycle number and the known optimal cycle number can correspond to an adjustment value. In certain aspects, for a particular PCR run, the algorithm can determine the optimal cycle number at which to stop a PCR reaction based on the adjustment value and the cycle number corresponding to the transition point. For example, the algorithm can determine the optimal cycle number by determining the transition point, identifying a cycle number corresponding to the transition point, and adding the adjustment value to the cycle number of the transition point.

In some examples, input data provided to the algorithm can include one or more cycles of the detected signals. Once the algorithm (e.g., the statistical model or AI model) receives the input parameters, the algorithm can use the detected signals to generate an output indicating the optimal cycle number. In some embodiments, the transition point is determined based on a zeroth derivative, a first derivative, and/or a second derivative of the detected signals. Additional higher-order derivatives may also be applicable to determine the transition point of the amplification reaction. In some embodiments, the transition point corresponds to a peak or a maximum point of the second derivative of the detected signals. For example, the transition point may be a local maximum or a global maximum of the second derivative. The peak of the second derivative can also be referred to as an inflection point. The inflection point can correspond to a point in a dataset of the detected signals at which curvature changes sign.

In some embodiments, the transition point corresponds to the zeroth derivative, the first derivative, and/or the second derivative of the function exceeding a set of one or more thresholds for each derivative type. In some implementations, the set of the thresholds may be determined based on historical data, training the algorithm, or a combination thereof. In some embodiments, the transition point corresponds to the second derivative of the function exceeding a threshold, and wherein a third derivative changes sign (e.g., from positive to negative or vice versa). In cases in which the second derivative has multiple peaks or local maxima, the transition point can be determined based on which peak of the second derivative exceeds the threshold. In some embodiments, the transition point is determined when an amplification efficiency rate determined from the detected signals is consistently positive (e.g., positive for a predefined number of cycles) and within an expected range. The amplification efficiency rate can correspond to a rate at which the detected signals are changing above a particular baseline or threshold. The amplification efficiency rate being positive can correspond to an amount of amplification product increasing, rather than decreasing or staying the same. In some examples, the amplification efficiency rate can be determined by applying a second derivative to the dataset of the detected signals.

For a typical PCR curve, identifying a transition point at the end of the initial portion of the amplification reaction, which is commonly referred to as an elbow value or a cycle threshold (Ct) value, is useful for understanding characteristics of the PCR amplification process. The Ct value may be used as a measure of the progress of the PCR process. For example, typically a defined signal threshold is determined for all reactions to be analyzed and the number of cycles (Ct) required to reach this threshold value is determined for a target nucleic acid as well as for reference nucleic acids such as a standard or housekeeping gene.

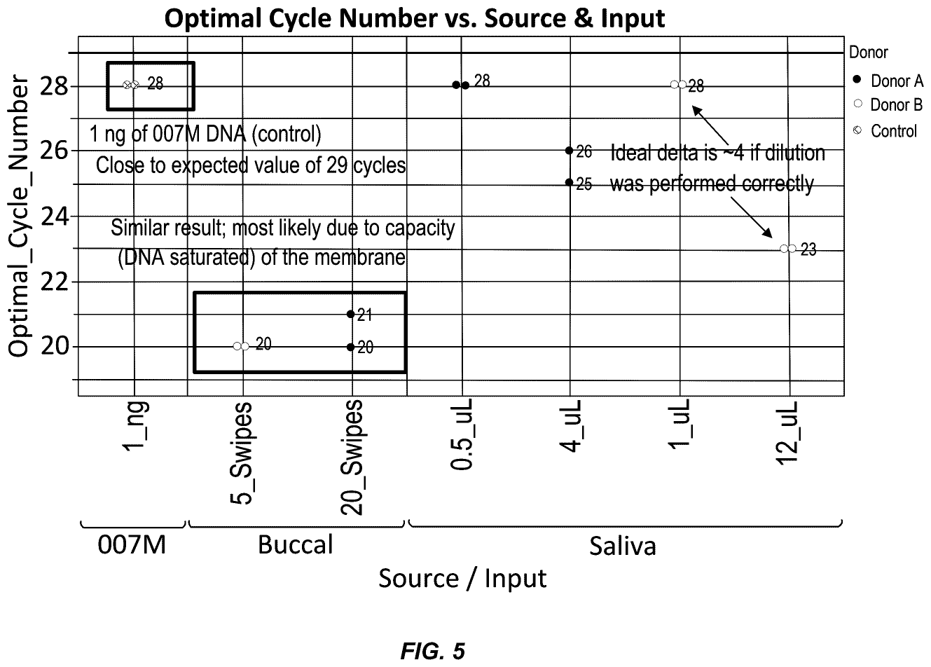

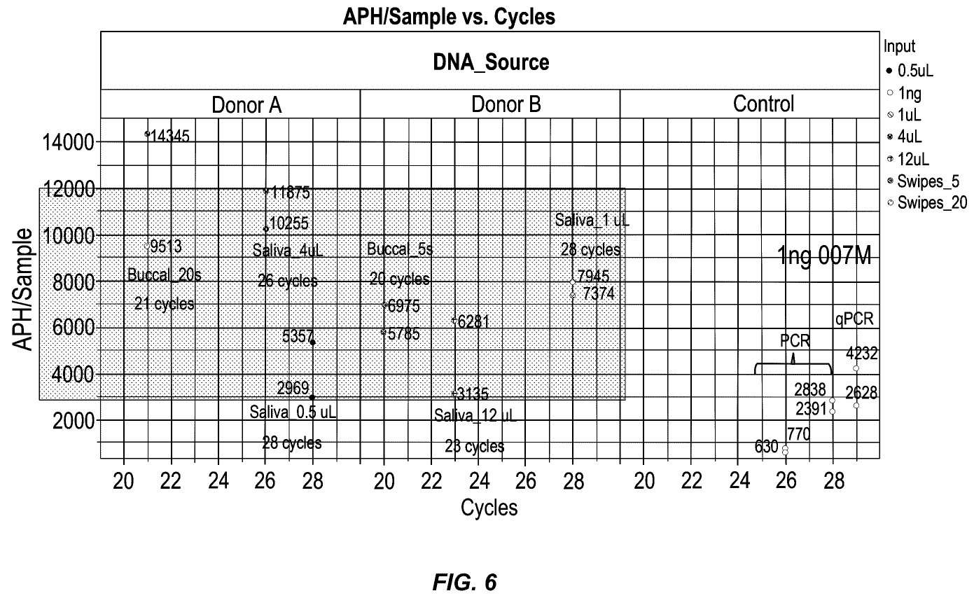

In certain aspects, the optimal cycle number determined using the algorithm described herein can be verified. In particular, amplification products (e.g., amplicons) produced by PCR stopped at an optimal cycle number can be quantified to ensure that the amplification products are within a suitable range. In some implementations, separate sets of amplification reactions can be performed using the same samples to confirm that samples amplified to a corresponding optimal cycle number yield consistent and suitable peak heights. FIG. 5 illustrates equivalent cycle number prediction between replicates of the same samples of within +/−1 cycle. More specifically, FIG. 5 shows a plot of optimal cycle number (y-axis) versus sample type (x-axis). FIG. 6 illustrates equivalent average sample peak heights between replicates of the same samples of within +/−1 cycle. The samples shown in FIG. 6 correspond to the samples shown in FIG. 5. In particular, Donor A and Donor B each provided three separate samples that were split into two aliquots that were amplified separately. A 1 ng control sample was prepared and amplified similarly.

A first aliquot of each sample was amplified to produce amplification data (e.g., amplification signals versus cycle number) that was provided as input to a trained algorithm to determine an optimal cycle number for each sample. FIG. 5 shows the optimal cycle number outputted by the algorithm for each sample. In particular, FIG. 5 indicates that an optimal cycle number predicted for replicates of the same sample was within plus or minus one cycle. In other words, the algorithm described herein outputs optimal cycle numbers that are consistent (e.g., within a suitable range of discrepancy) when tested with replicates of the same sample. A second aliquot of each sample was amplified based on the optimal cycle number determined using the first aliquot of each sample. Once the second aliquot was amplified, amplification products produced by amplifying the second aliquot were quantified to determine a quantity of amplicons per sample. The quantity (y-axis) of each sample is provided in FIG. 6 for various cycles (x-axis). In general, results shown in FIG. 6 indicate that using the optimal cycle number to run amplification reactions can produce amplification products that are consistent in amplicon concentration and that have consistent peak locations and peak heights.

### II. METHOD

FIG. 7 is a flow chart of an example method 700 of determining an optimal amplification cycle number according to some embodiments of the present disclosure. Method 700 can be implemented by a computer system, such as computer system 800 described below with respect to FIG. 8. In some examples, the optimal amplification cycle number can correspond to a growth process, such as a polymerase chain reaction (PCR). Other growth processes include bacterial processes, enzymatic processes, or binding processes. Growth processes can be measured using one or more data points (e.g., a series of data points), with each data point providing a respective signal strength at a corresponding cycle number. In some embodiments, samples being amplified in a PCR as described herein may have an unknown DNA quantity. In other words, a quantification step to determine the DNA quantity of the samples may be omitted.

At block 702, one or more signals are detected. In some implementations, the signals can be detected once PCR is initiated. The signals (e.g., a strength or a magnitude of the signals) can represent amplification products at each of n cycles of an amplification reaction. The amplification products can correspond to a sample (e.g., a DNA segment or a genetic sequence) being amplified or copies of the amplified sample. In some examples, each signal can correspond to a respective data point of a dataset that can include one or more data points. Each data point can indicate a signal strength at a corresponding cycle number of the amplification reaction. For example, datasets of FIGS. 3A and 3B can include data points corresponding to a particular fluorescence intensity and a particular cycle number. In some aspects, the signals can be detected over a time window, such as seconds, minutes, or hours. The time window can be a predefined time window, for example based on a particular number of cycles. For example, the amplification reaction may be monitored for a predefined number of cycles (e.g., 15 cycles) to collect data prior to predicting the optimal cycle number. In some cases, one PCR cycle may take about one minute to be completed.

As described herein, a sensor system can be provided to detect the signals of a PCR run. In some cases, the sensor system can be referred to as a sensing module. The sensor system can include one or more sensors positioned in a reaction chamber in which PCR is performed. In some examples, the sensors may be part of instrumentation used to perform the PCR run. Although certain aspects of the present disclosure are described herein with respect to fluorescence signals, it will be appreciated that other quantification techniques are possible. Non-limiting examples of the quantification techniques include determining a quantity of the amplified sample using conductivity, concentration, pH, temperature, current, voltage, weight, mass, or a combination thereof.

At block 704, amplification is monitored based on the detected signals as a function of cycle number. As described herein, the detected signals can indicate an amount of amplification products corresponding to a particular cycle number. Accordingly, a rate at which the sample is being amplified can be determined using the detected signals. In some examples, as shown in FIG. 3A and FIG. 3B, the amplification of the sample may initially have a relatively low amplification rate such that a magnitude of the detected signals remains approximately the same. In some embodiments, this initial portion of the amplification can function as a baseline or a comparison that is referenced to determine where the amplification rate significantly increases.

At block 706, a transition point of the amplification is determined. The transition point can correspond to a point at which the amplification rate significantly increases. A computer system (e.g., computer system 800) may receive the detected signals from the sensor system, such as periodically (e.g., at particular time intervals) or in real-time. The computer system can provide the detected signals as an input to an algorithm that can determine a transition point of the dataset of the detected signals. As described herein, the transition point can correspond to a change in the detected signals. More specifically, the transition point can correspond to a change in the detected signals outside of a predefined range (e.g., with respect to the baseline of the amplification).

In some examples, the transition point can be determined by analyzing the dataset of the detected signals using one or more derivatives (e.g., a zeroth derivative, a first derivative, a second derivative, a third derivative, etc.). The dataset of the detected signals can be differentiated repeatedly, such as using higher-order derivatives (e.g., a second derivative, third derivative, etc.). In some embodiments, a zeroth derivative can correspond to a particular data point of the dataset. In some embodiments, a first derivative of the dataset can correspond to a slope of a function associated with the dataset. In some embodiments, a second derivative of the dataset can correspond to a rate at which the first derivative is changing, for example a rate of change of the slope of the function associated with the dataset. In some embodiments, a third derivative of the dataset can correspond to a rate at which the second derivative is changing.

In some examples, the transition point can be determined based on a particular derivative of the dataset exceeding a predefined threshold. Each derivative type (e.g., zeroth, first, second, third, etc.) can have a respective set of one or more thresholds. In general, a derivative can be applied to determine a rate of change or a ratio of change between a dependent variable (e.g., the detected signals) and an independent variable (e.g., cycle number). Accordingly, the derivative(s) can be applied to determine the change in the detected signals. For example, a first derivative can indicate a rate of change in the detected signals over a number of cycles. A second derivative can indicate how quickly the detected signals are increasing above a baseline of the amplification. In some implementations, the baseline can correspond to the initial portion of the amplification for which the sensor system is unable to detect the signals of the amplification. As another example, at a peak (e.g., a local maximum) of a second derivative, the amplification can begin to decelerate, such as due to reduced molecular resources (e.g., resources that are needed to sustain the amplification reaction).

In some embodiments, a statistical model (e.g., a regression model or a fitting model) or another suitable mathematical model can be used to process the detected signals. For example, the detected signals can be provided as an input to the statistical model. The statistical model can identify the transition point based on the input. In some embodiments, the transition point can be determined using a regression model based on a coefficient of a regression function associated with the regression model. When the coefficient changes by an amount greater than a threshold, the coefficient can be determined to correspond to the transition point. In some embodiments, the transition point can be determined based on a fitting score. The fitting score can also be referred to as a similarity score. For example, each detected signal can have or be associated with a respective fitting score. When a particular detected signal has a corresponding fitting score larger than a statistical parameter (e.g., an average, a median, a mode, etc.), the particular detected signal can be determined to correspond to the transition point. In some examples, the statistical parameter can be determined based on a current dataset of the detected signals that can be updated as additional detected signals are obtained by the sensor system.

In some implementations, the transition point can be determined based on a baseline of the amplification. In particular, the transition point can correspond to a point of the amplification at which the amplification has significantly increased above the baseline, such as above a predefined threshold. In some cases, the baseline can be determined based on one or more patterns in a zeroth derivative, a first derivative, and/or a second derivative of the detected signals. Historical data (e.g., signals or datasets from previous PCR runs) may be analyzed to determine the patterns used to determine the baseline. Additionally or alternatively, the baseline can be determined by a machine learning approach or an artificial intelligence approach. For example, the machine learning approach or the artificial intelligence approach may involve pattern recognition of regularities or patterns in the detected signals to enable a machine or computer program to determine the baseline. As described herein, the machine learning approach or the artificial intelligence approach can involve training a computer program, algorithm, or model using one or more suitable techniques based on historical data or other suitable datasets. In some embodiments, the method further comprises determining an initial baseline based on a subset of the detected signals corresponding to a predefined number of the n cycles. In other words, the algorithm may use the detected signals corresponding to a predefined number of cycles to determine the initial baseline. As a non-limiting example, the algorithm may gather a subset of the detected signals corresponding to an initial ten PCR cycles and determine the initial baseline based on the subset of the detected signals.

In some embodiments, the method further comprises, in response to receiving an updated set of the detected signals, comparing the updated set of the detected signals to the initial baseline. For example, the computer system may receive at least one additional signal detected subsequent to the initial ten PCR cycles. The additional signal can be part of the updated set of the detected signals. The computer system then can compare the additional signal to the initial baseline to determine whether to update the initial baseline based on whether the additional signal is consistent with the initial baseline. The additional signal may be consistent with the initial baseline if a magnitude of the additional signal is within a predefined range (e.g., plus or minus 20%).

In some embodiments, if the updated set of the detected signals is consistent with the initial baseline, the initial baseline is updated to include the updated set of the detected signals. In other words, an updated baseline can be generated by updating the initial baseline to include the additional signal. The updated baseline then can be used as a comparison for subsequent detected signals to identify the transition point of the amplification. In some embodiments, if the updated set of the detected signals exceeds the initial baseline, the transition point is determined to correspond to a particular cycle number of the updated set of the detected signals. For example, if the additional signal has a magnitude outside of the predefined range, the computer system may determine that a cycle number corresponding to the additional signal is the transition point. As another example, if the additional signal has a magnitude outside of the predefined range, the computer system may continue to compare one or more subsequent detected signals received from the sensor system to the initial baseline or an updated baseline prior to determining the transition point. The subsequent detected signals can be used to confirm whether the additional signal is the transition point or an outlier.

At block 708, an optimal cycle number m can be predicted based on the transition point. In particular, the transition point can anticipate the optimal cycle number m by a predefined number of cycles. As described herein, such as with respect to FIG. 4A and FIG. 4B, the algorithm used to determine the optimal cycle number can generate a consistent difference between the optimal cycle number m and a cycle number corresponding to the transition point. Through training or data analysis, an adjustment value can be determined based on the difference between the optimal cycle number m and the cycle number corresponding to the transition point. In particular, the adjustment value can be added to the cycle number of the transition point to determine the optimal cycle number m. Accordingly, based on the transition point, the computer system can transmit a control signal or otherwise indicate to stop the amplification reaction at the optimal cycle number m prior to the amplification reaction arriving at the optimal cycle number m. In some examples, the optimal cycle number is larger than a current number of cycles of the amplification reaction. The algorithm of the computer system can be trained to use the detected signals as an input and output an indication of the optimal cycle number m for the sample being amplified based on the detected signals.

As indicated above, in some embodiments, the optimal amplification cycle number m is determined by adding an adjustment value to a cycle number corresponding to the transition point of the amplification. The adjustment value may be an integer (e.g., a whole number) or a non-integer (e.g., a decimal). In some embodiments, the adjustment value is predefined based on the determination of a transition point. More specifically, the adjustment value may vary depending on how the transition point is determined. In some embodiments, m is equal to or more than n+1. In some embodiments, m equals to n+1, n+2, n+3, n+4, or n+5. In some embodiments, n is an integer at least one. In some embodiments, n is between one to twenty. For example, n may be one, two, three, four, five, six, seven, eight, nine, ten, eleven, twelve, thirteen, fourteen, fifteen, sixteen, seventeen, eighteen, nineteen, twenty, or anywhere in between. In some embodiments, n is between five to ten.

As a non-limiting example, an example PCR growth curve 305 shown in FIG. 3A has a transition point 310 corresponding to a cycle number of twenty. Amplification signals used to generate example PCR growth curve 305 can be provided to an algorithm to determine an optimal cycle number. Based on the amplification signals, the algorithm can determine that transition point 310 corresponds to the twentieth cycle of the amplification reaction. As an example, the algorithm may use amplification signals from the first fifteen cycles of the amplification reaction to determine an initial baseline. Each subsequent amplification signal can be compared to a baseline 315 (e.g., an initial baseline or an updated baseline) to determine whether the subsequent amplification signal is consistent with baseline 315. For example, based on an amplification signal of the sixteenth cycle being consistent with the initial baseline, the initial baseline can be updated to account for the amplification signal of the sixteenth cycle, thereby generating the updated baseline. Once the amplification signal corresponding to the twentieth cycle of the amplification reaction was received, the amplification signal can be compared to baseline 315. Transition point 310 can be determined based on the amplification signal of the twentieth cycle exceeding baseline 315. Once transition point 310 is determined, an adjustment value can be added to the cycle number corresponding to transition point 310 to determine the optimal cycle number. For example, if the adjustment value was five, the optimal cycle number of example PCR growth curve 305 would be 25. Optimal point 320 indicates a point on example PCR growth curve 305 corresponding to the optimal cycle number.

### III. COMPUTER SYSTEM

Any of the computer systems mentioned herein may utilize any suitable number of subsystems. Examples of such subsystems are shown in FIG. 8 in computer system 800. In some embodiments, a computer system includes a single computer apparatus, where the subsystems can be the components of the computer apparatus. In other embodiments, a computer system can include multiple computer apparatuses, each being a subsystem, with internal components. In some embodiments, computer system 800 can be in communication (e.g., wireless communication via a network) with PCR instrumentation used to perform or monitor a PCR run. In some embodiments, computer system 800 can be a component of or be integrated with the PCR instrumentation. In some embodiments, computer system 800 can receive data (e.g., detected signals) from the PCR instrumentation. Additionally, computer system 800 can transmit commands to the PCR instrumentation to control the PCR run, such as to stop the PCR run.

The subsystems shown in FIG. 8 are interconnected via a system bus 875. Additional subsystems such as a printer 874, keyboard 878, storage device(s) 879, monitor 876, which is coupled to display adapter 882, and others are shown. Peripherals and input/output (I/O) devices, which couple to I/O controller 871, can be connected to the computer system by any number of means known in the art, such as serial port 877. For example, serial port 877 or external interface 881 (e.g. Ethernet, Wi-Fi, etc.) can be used to connect computer system 800 to a wide area network such as the Internet, a mouse input device, or a scanner. The interconnection via system bus 875 allows the central processor 873 to communicate with each subsystem and to control the execution of instructions from system memory 872 or the storage device(s) 879 (e.g., a fixed disk), as well as the exchange of information between subsystems. The system memory 872 and/or the storage device(s) 879 may embody a computer readable medium. Any of the values mentioned herein can be output from one component to another component and can be output to the user.

A computer system can include a plurality of the same components or subsystems, e.g., connected together by external interface 881 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware (e.g. an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer program product (e.g. a hard drive, a CD, or an entire computer system), and may be present on or within different computer program products within a system or network. A computer system may include a monitor, printer, or other suitable display or output device for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

In the preceding description, various embodiments have been described. For purposes of explanation, specific configurations and details have been set forth in order to provide a thorough understanding of the embodiments. However, it will also be apparent to one skilled in the art that the embodiments may be practiced without the specific details. Furthermore, well-known features may have been omitted or simplified in order not to obscure the embodiment being described. While example embodiments described herein center on using optical spectroscopy to monitor an amplification reaction, these are meant as non-limiting, illustrative embodiments. Embodiments of the present disclosure are not limited to such techniques, but rather are intended to address amplification reaction systems for which a wide array of analysis techniques can be applied to monitor the amplification reaction occurring in these systems. Such analysis techniques may include, but are not limited to, quantifying conductivity, current, voltage, weight, or a combination thereof.

Some embodiments of the present disclosure include a system including one or more data processors and/or logic circuits. In some embodiments, the system includes a non-transitory computer readable storage medium containing instructions (e.g., executable instructions, one or more computer programs, or one or more applications) which, when executed on the one or more data processors, cause the one or more data processors to perform part or all of one or more methods and/or part or all of one or more processes and workflows disclosed herein. Some embodiments of the present disclosure include a computer-program product tangibly embodied in a non-transitory machine-readable storage medium, for example, in the form of a computer program including a plurality of instructions executable by one or more processors. The instructions can be configured to cause one or more data processors to perform part or all of one or more methods and/or part or all of one or more processes disclosed herein, including, for example, method 700 of FIG. 7.

The terms and expressions which have been employed are used as terms of description and not of limitation, and there is no intention in the use of such terms and expressions of excluding any equivalents of the features shown and described or portions thereof, but it is recognized that various modifications are possible within the scope of the claims. Thus, it should be understood that although the present disclosure includes specific embodiments and optional features, modification and variation of the concepts herein disclosed may be resorted to by those skilled in the art, and that such modifications and variations are considered to be within the scope of the appended claims.

The present disclosure will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### IV. EXAMPLES

### Example 1: Materials and Methods

### 1. DNA Sample Preparation

Control DNA 007 (Thermo Fisher Scientific Inc, Waltham, MA) at a starting concentration of 100 ng/μL was used. 2-fold and 10-fold DNA dilutions series were prepared. Stock DNA was quantified using Nanodrop (Thermo Fisher Scientific Inc, Waltham, MA) prior to setting up serial dilutions. DNA dilutions were prepared by serially diluting stock DNA in DNA suspension buffer (10 mM Tris-HCl pH 8.0 and 0.1 mM EDTA, Teknova Inc, Hollister, CA). 10-fold serial dilution consisted of final DNA inputs in PCR of 100 ng, 10 ng, 1 ng, and 0.1 ng. 2-fold serial dilution consisted of final DNA inputs in PCR of 16 ng, 8 ng, 4 ng, 2 ng, and 1 ng.

### 2. Taqman™ Assay for Sensing DNA Amplification at Real Time

Primers and TaqMan™ probe were designed for a multicopy human target with a fragment size of 214 bp to match the median size of the GlobalFiler™ IQC PCR Amplification Kit (Thermo Fisher Scientific Inc, Waltham, MA) amplicons. The TaqMan™ probe utilizes the Cy5.5 fluorescent dye (Thermo Fisher Scientific Inc, Waltham, MA), paired with a QSY™ 21 quencher (Thermo Fisher Scientific Inc, Waltham, MA).

### 3. PCR Amplification and Thermal Cycling Conditions

PCR amplification was performed for all DNA input levels using the GlobalFiler™ IQC PCR Amplification Kit spiked with primers and probes for the new TaqMan™ assay. GlobalFiler™ assay components (Primer Set and Master Mix) were added per instructions in the kit's user guide. TaqMan™ assay primers and probe were added at a 600 nM and 300 nM concentration respectively. Final reaction volume was set to 25 μL, including addition of appropriate amounts of sample DNA.

PCR was setup on a QuantStudio™ 5 Real-Time PCR system (Thermo Fisher Scientific Inc, Waltham, MA). The GlobalFiler™ IQC kit thermal cycling protocol was setup on the QuantStudio™ Design and Analysis software as a custom assay per instructions in the user guide. For real time TaqMan™ assay signal acquisition, only filter 6 was turned on for excitation and emission (editable in the ‘Method’ tab of the assay setup) corresponding to the spectrum of the CY5.5 dye, to minimize excitation and emission from the GlobalFiler™ assay dyes that would result in dye photobleaching as well as background saturation, negatively impacting the TaqMan™ assay. PCR was run for 29 cycles.

### Example 2: Sensitivity Series Training Data for Real Time Amplification Sensing Algorithm

We utilized the PCR modules and sensors in Applied Biosystems QuantStudio™ 5 to develop sensing algorithms. Real time cycle-by-cycle signals were detected and fed to the algorithms. The algorithms were trained using known optimal cycle numbers on a range of sample type and sample concentration to predict optimal cycle numbers of unknown samples. Specifically, second derivatives of each curve of unknown sample are used for baselining and recognizing transition points. These transition points occur a few cycles ahead of the optimal cycle numbers.

For standard real time PCR data, a 10-fold DNA serial dilution would result in an ˜3 cycle Ct shift and a 2-fold DNA serial dilution would result in a 1 cycle Ct shift between adjacent inputs. Results with the new the TaqMan™ assay (coamplified with the GlobalFiler™ assay) show real time curves for various DNA inputs that are separated from each other by the expected cycle numbers. As shown in FIG. 3A, an amplification initiated with 100 ng, 10 ng, 1 ng, or 0.1 ng of DNA template reached to a transition point at 20, 23, 26, or >29 cycles, respectively. As shown in FIG. 3B, an amplification started with 16 ng, 8 ng, 4 ng, 2 ng or 1 ng of DNA template reached to a transition point at 22, 23, 24, 25 or 26 cycles, respectively. This demonstrates the robustness of the assay to detect down to 2-fold differences in DNA concentrations being amplified.

### Example 3: Real Time Amplification Sensing Algorithm Training for Prediction of Breakout Cycle Number

### 1. DNA Sample Preparation

Control DNA 007 (Thermo Fisher Scientific Inc, Waltham, MA) at a starting concentration of 100 ng/μL was used. Stock DNA was quantified using Nanodrop (Thermo Fisher Scientific Inc, Waltham, MA) prior to setting up serial dilutions. DNA dilutions were in DNA suspension buffer (10 mM Tris-HCl pH 8.0 and 0.1 mM EDTA, Teknova Inc, Hollister, CA).

In addition to DNA serial dilutions described above, additional samples were prepared with Control DNA 007 containing various PCR inhibitors. Total DNA input in PCR was fixed at either 16 ng or 1 ng. Inhibitor levels tested in PCR were 100-200 ng/μL humic acid, 200-400 μM hematin, and 5-50 mg soil.

### 2. Comparison of Optimal Cycle Number Generated by Real Time Amplification Sensing Algorithms

Real time qPCR curves were generated from amplification DNA samples of known inputs and inhibitor concentrations using the TaqMan™ assay (coamplified with the GlobalFiler™ assay). This data was then fed to two versions of algorithms (CtProxy and EffLoc) for analysis and estimation of when the real time PCR curve breaks away from the baseline to reach the exponential phase of amplification, for each DNA sample. The algorithm generated breakout cycle number was then compared to a known optimal stop cycle number for each DNA sample, and a difference was calculated. A plot of the difference in cycle numbers for each sample was then generated for each algorithm evaluated, as shown in FIG. 4A for the algorithm Ctproxy and FIG. 4B for the algorithm EffLoc.

The results show that across a range of DNA sample inputs (with or without varying levels of inhibition), both algorithms (CtProxy and EffLoc) generated a consistent difference between the breakout cycle number and optimal stop cycle number. For the CtProxy method (FIG. 4A), the delta was established to be 1.5 (+/−0.5) cycles. For the EffLoc method (FIG. 4B), the delta was established to be 5 (+/−1) cycles. This suggests that both the algorithms consistently detect a presence of DNA amplification, and thereby sufficient DNA concentration, prior to when the PCR would have to be stopped for a given DNA input to generate optimal PCR results for downstream applications. This established delta for known sample inputs is then fed to train the algorithm to determine optimal PCR cycle numbers.

### Example 4: Verification of Algorithmic Estimation of Optimal PCR Cycle Number

This example illustrates how we verify the method of algorithmic estimation of optimal PCR cycle number using target genes and house-keeping genes. In brief, we designed primers to amplify target genes and house-keeping genes. The quantity ratio between each genes remained constant during amplification processes. We used existing Taqman technology to detect the quantity of human house-keeping genes during PCR process as a proxy to represent the amplification quantities of target genes. Then we collected the signals and ran the algorithms and stopped the PCRs at the optimal cycle numbers provided by the trained algorithms. We collected the PCR products and ran DNA fragment analysis using capillary electrophoresis. The peak location and height on electropherogram from electrophoresis represented the fragment sizes and amounts. We showed that using our approach, the peak locations and peak heights were highly consistent regardless of sample types and concentrations. DNA samples between 4 and 100 ng were amplified and all target DNA amplicons fell within 4 times amplicon concentration with all peaks correctly called out. This method is a major improvement from traditional PCR approach that results in 25 times amplicon concentration difference and false positive and false negative of peak call-out.

### 1. DNA Sample Preparation

Control DNA 007 (Thermo Fisher Scientific Inc, Waltham, MA) at a starting concentration of 100 ng/μL was used. Stock DNA was quantified using Nanodrop (Thermo Fisher Scientific Inc, Waltham, MA) prior to setting up serial dilutions. DNA dilutions were in DNA suspension buffer (10 mM Tris-HCl pH 8.0 and 0.1 mM EDTA, Teknova Inc, Hollister, CA).

Biological samples of unknown DNA quantity were collected either as buccal swabs or saliva spotted on cotton swabs (Puritan Medical Products Company LLC, Guilford, ME). Buccal swab and saliva samples were collected from the same donor. Buccal swabs at 2 input levels were collected as 5× or 20×cheek swipes using cotton swabs and air dried overnight prior to use. Saliva samples were diluted in DNA suspension buffer and an amount equivalent to 0.5 μL, 1 μL, 4 μL, or 12 μL, of saliva was spotted onto cotton swabs and air dried overnight prior to use. DNA from the samples was extracted using the PrepFiler™ Forensic DNA Extraction chemistry ((Thermo Fisher Scientific Inc, Waltham, MA). Sample lysates were initially pooled to minimize swab to swab variability and the lysate was then split into 4 aliquots for sample preparation. The extracted DNA was then collected on a 3 mm sized punch of Whatmann™ glass microfiber membrane (Cytivia Life Sciences, Marlborough, MA). 1 ng Control DNA 007 samples were also amplified as control reactions with known DNA input.

### 2. PCR Amplification and Thermal Cycling Conditions

PCR amplification was performed as previously described with a final reaction volume of 25 μL. For these experiments, sample addition included a 3 mm glass microfiber membrane punch containing extracted DNA from biological samples to each reaction well. PCR was setup on a QuantStudio™ 5 as previously described.

### 3. Determination of Optimal Cycle Number by Algorithm

Two out of four replicates of DNA extracted on glass microfiber membrane, for each sample type and input quantity, were amplified on the QuantStudio™ using the TaqMan™ assay (coamplified with the GlobalFiler™ assay) for 29 cycles to generate full real time PCR curves. This data was then fed to the algorithm to suggest an optimal cycle number for each set of samples. The remaining two replicates of extracted DNA for each sample type were then run on QuantStudio™ using the TaqMan™ assay (coamplified with the GlobalFiler™ assay) with PCR stopping at the optimal cycle numbers predicted by the algorithm. PCR amplification products were then run on the 3500xL Genetic Analyzer (Thermo Fisher Scientific Inc, Waltham, MA) to confirm optimal assay performance.

As shown in FIG. 5, for the first set of amplifications used to predict optimal PCR cycle numbers using the algorithm, results show equivalent cycle number prediction between replicates of the same sample of within +/−1 cycle. Likewise, as shown in FIG. 6, the second set of replicates, for each sample, amplified at their determined optimal cycle numbers yield quality profiles with equivalent average sample peak heights between replicates. The sample peak heights also fall within the expected optimal peak height range for the assay. All other profile quality metrics were also met for all samples. The higher peak height variability observed between replicates of some samples is not unexpected. Only variability coming from the sample lysis step was eliminated by pooling the lysate, but all other sources of variability such as individual DNA extractions, PCR and capillary electrophoresis runs remain.

Overall, these results demonstrate the robustness of this new approach to monitor amplification of biological samples of unknown DNA quantity in real time and determine optimal PCR cycle number based on template DNA concentration to yield desired results for downstream interpretation.

### V. ILLUSTRATIVE ASPECTS

In the following sections, further exemplary embodiments are provided.

Aspect 1 includes a method of determining an optimal amplification cycle number m for an amplification reaction of a template nucleic acid of unknown copy number in a sample, the method comprising: detecting signals representing amplification products at each cycle of n cycles of the amplification reaction; monitoring, based on the detected signals, the amplification reaction as a function of cycle number; determining a transition point of the amplification reaction, wherein the transition point corresponds to a change in the detected signals; and predicting the optimal amplification cycle number m based on the transition point, wherein m is larger than n.

Aspect 2 includes a method of any of aspect(s) 1, further comprising: determining an initial baseline based on a subset of the detected signals corresponding to a predefined number of the n cycles; in response to receiving an updated set of the detected signals, comparing the updated set of the detected signals to the initial baseline; if the updated set of the detected signals is consistent with the initial baseline, updating the initial baseline to include the updated set of the detected signals; and if the updated set of the detected signals exceeds the initial baseline, determining that the transition point corresponds to a particular cycle number of the updated set of the detected signals.

Aspect 3 includes a method of any of aspect(s) 1-2, wherein the initial baseline is determined based on one or more patterns in a zeroth derivative, a first derivative, and/or a second derivative of the detected signals.

Aspect 4 includes a method of any of aspect(s) 1-3, wherein the initial baseline is determined by a machine learning approach or an artificial intelligence approach.

Aspect 5 includes a method of any of aspect(s) 1-4, wherein the transition point is determined based on a zeroth derivative, a first derivative, and/or a second derivative of the detected signals.

Aspect 6 includes a method of any of aspect(s) 1-5, wherein the transition point corresponds to a peak of the second derivative of the detected signals.

Aspect 7 includes a method of any of aspect(s) 1-6, wherein the transition point corresponds to the zeroth derivative, the first derivative, and/or the second derivative of the function exceeding a set of one or more thresholds for each derivative type.

Aspect 8 includes a method of any of aspect(s) 1-7, wherein the transition point corresponds to the second derivative of the function exceeding a threshold, and wherein a third derivative changes sign.

Aspect 9 includes a method of any of aspect(s) 1-8, wherein the transition point is determined when an amplification efficiency rate determined from the detected signals is consistently positive and within an expected range.

Aspect 10 includes a method of any of aspect(s) 1-9, wherein n is an integer at least 1.

Aspect 11 includes a method of any of aspect(s) 1-10, wherein m is determined by adding an adjustment value to a cycle number corresponding to the transition point of the amplification reaction.

Aspect 12 includes a method of any of aspect(s) 1-11, wherein the adjustment value is predefined based on the determination of the transition point.

Aspect 13 includes a method of any of aspect(s) 1-12, wherein m is equal o or more than n+1.

Aspect 14 includes a method of any of aspect(s) 1-13, wherein the template nucleic acid has a concentration in a range of 0.001-500 ng per 25 μL, optionally in a range of 0.1-100 ng per 25 μL.

Aspect 15 includes a method of any of aspect(s) 1-14, wherein the amplification reaction is a Taqman PCR.

Aspect 16 includes a method of any of aspect(s) 1-15, wherein the predicting step comprises: providing the detected signals as input to an artificial intelligence (AI) model, wherein the AI model optionally comprises a machine-learning model; and executing, based on the detected signals, the AI model to generate an output indicating the optimal amplification cycle number m.

Aspect 17 includes a method of any of aspect(s) 1-16, further comprising, prior to the detecting step: training a computer program using one or more training samples with known optimal amplification cycle numbers.

Aspect 18 includes a method of any of aspect(s) 1-17, wherein the optimal amplification cycle number m is predicted by the computer program using one or more cycles of the detected signals.

Aspect 19 includes a method of amplifying one or more target genes in a sample, comprising: performing an amplification reaction using the sample; determining an optimal amplification cycle number m using the method of any one of aspects 1-18; and stopping the amplification reaction at the optimal amplification cycle number m.

Aspect 20 includes a method of any of aspect(s) 1-19, wherein the amplification reaction comprises amplifying a housekeeping gene simultaneously with the one or more target genes in the same sample.

Aspect 21 includes a computer product comprising a non-transitory computer readable medium storing a plurality of instructions to perform one or more operations of a method described in or related to any of the preceding aspects that, when executed, control a computer system to determine an optimal amplification cycle number m for an amplification reaction of a template nucleic acid of unknown copy number in a sample.

Aspect 22 includes a Polymerase Chain Reaction (PCR) system, comprising: a PCR data acquiring device configured to detect signals representing amplification products at each cycle of n cycles of an amplification reaction; and a computer system configured to process the signals to determine an optimal amplification cycle number m the amplification reaction of a template nucleic acid of unknown copy number in a sample by performing one or more operations of a method described in or related to any of the preceding examples.

The description provides exemplary embodiments, and is not intended to limit the scope, applicability or configuration of the disclosure. Rather, the ensuing description of the exemplary embodiments will provide those skilled in the art with an enabling description for implementing various embodiments. It is understood that various changes may be made in the function and arrangement of elements without departing from the spirit and scope as set forth in the appended claims.

Specific details are given in the description to provide a thorough understanding of the embodiments. However, it will be understood that the embodiments may be practiced without these specific details. For example, specific system components, systems, processes, and other elements of the present disclosure may be shown in schematic diagram form or omitted from illustrations in order not to obscure the embodiments in unnecessary detail. In other instances, well-known circuits, processes, components, structures, and/or techniques may be shown without unnecessary detail.

What is claimed is:

1. A method of determining an optimal amplification cycle number m for an amplification reaction of a template nucleic acid of unknown copy number in a sample, the method comprising:
detecting signals representing amplification products at each cycle of n cycles of the amplification reaction;
monitoring, based on the detected signals, the amplification reaction as a function of cycle number;
determining a transition point of the amplification reaction, wherein the transition point corresponds to a change in the detected signals; and
predicting the optimal amplification cycle number m based on the transition point, wherein m is larger than n.

2. The method of claim 1, further comprising:
determining an initial baseline based on a subset of the detected signals corresponding to a predefined number of the n cycles;
in response to receiving an updated set of the detected signals, comparing the updated set of the detected signals to the initial baseline;
if the updated set of the detected signals is consistent with the initial baseline, updating the initial baseline to include the updated set of the detected signals; and
if the updated set of the detected signals exceeds the initial baseline, determining that the transition point corresponds to a particular cycle number of the updated set of the detected signals.

3. The method of claim 2, wherein the initial baseline is determined based on one or more patterns in a zeroth derivative, a first derivative, and/or a second derivative of the detected signals.

4. The method of claim 1, wherein the transition point is determined based on a zeroth derivative, a first derivative, and/or a second derivative of the detected signals.

5. The method of claim 4, wherein the transition point corresponds to a peak of the second derivative of the detected signals.

6. The method of claim 4, wherein the transition point corresponds to the zeroth derivative, the first derivative, and/or the second derivative of the function exceeding a set of one or more thresholds for each derivative type.

7. The method of claim 4, wherein the transition point corresponds to the second derivative of the function exceeding a threshold, and wherein a third derivative changes sign.

8. The method of claim 1, wherein the transition point is determined when an amplification efficiency rate determined from the detected signals is consistently positive and within an expected range.

9. The method of claim 1, wherein m is determined by adding an adjustment value to a cycle number corresponding to the transition point of the amplification reaction.

10. The method of claim 9, wherein the adjustment value is predefined based on the determination of the transition point.

11. The method of claim 1, wherein the template nucleic acid has a concentration in a range of 0.001-500 ng per 25 μL.

12. The method of claim 1, wherein the predicting step comprises:
providing the detected signals as input to an artificial intelligence (AI) model, wherein the AI model optionally comprises a machine-learning model; and
executing, based on the detected signals, the AI model to generate an output indicating the optimal amplification cycle number m.

13. The method of claim 1, further comprising, prior to the detecting step:
training a computer program using one or more training samples with known optimal amplification cycle numbers, wherein the optimal amplification cycle number m is predicted by the computer program using one or more cycles of the detected signals.

14. The method of claim 1, further comprising:
stopping the amplification reaction at the optimal amplification cycle number m.

15. The method of claim 1, wherein the amplification reaction comprises amplifying a housekeeping gene simultaneously with one or more target genes in the sample.

16. A computer product comprising a non-transitory computer readable medium storing a plurality of instructions to perform operations that, when executed, control a computer system to determine an optimal amplification cycle number m for an amplification reaction of a template nucleic acid of unknown copy number in a sample, the operations comprising:
detecting signals representing amplification products at each cycle of the n cycles of the amplification reaction;
monitoring, based on the detected signals, the amplification reaction as a function of cycle number;
determining a transition point of the amplification reaction, wherein the transition point corresponds to a change in the detected signals; and
predicting the optimal amplification cycle number m based on the transition point, wherein m is larger than n.

17. The computer product of claim 16, wherein the operations further comprise:
determining an initial baseline based on a subset of the detected signals corresponding to a predefined number of the n cycles;
in response to receiving an updated set of the detected signals, comparing the updated set of the detected signals to the initial baseline;
if the updated set of the detected signals is consistent with the initial baseline, updating the initial baseline to include the updated set of the detected signals; and
if the updated set of the detected signals exceeds the initial baseline, determining that the transition point corresponds to a particular cycle number of the updated set of the detected signals.

18. The computer product of claim 17, wherein the initial baseline is determined based on one or more patterns in a zeroth derivative, a first derivative, and/or a second derivative of the detected signals.

19. A Polymerase Chain Reaction (PCR) system, comprising:
a PCR data acquiring device configured to detect signals representing amplification products at each cycle of n cycles of an amplification reaction; and
a computer system configured to process the signals to determine an optimal amplification cycle number m for the amplification reaction of a template nucleic acid of unknown copy number in a sample by:
receiving the detected signals;
monitoring, based on the detected signals, the amplification reaction as a function of cycle number;
determining a transition point of the amplification reaction, wherein the transition point corresponds to a change in the detected signals; and
predicting the optimal amplification cycle number m based on the transition point, wherein m is larger than n.

20. The PCR system of claim 19, wherein the transition point is determined by:
determining an initial baseline based on a subset of the detected signals corresponding to a predefined number of the n cycles;
in response to receiving an updated set of the detected signals, comparing the updated set of the detected signals to the initial baseline;
if the updated set of the detected signals is consistent with the initial baseline, updating the initial baseline to include the updated set of the detected signals; and
if the updated set of the detected signals exceeds the initial baseline, determining that the transition point corresponds to a particular cycle number of the updated set of the detected signals.
